(19) United States
(12) Patent Application Publication
(10) Pub. No.: US 2004/0102832 A1
(43) Pub. Date: May 27, 2004

(54) Stent delivery and retention apparatus

(76) Inventor: David R. Doty, Forestville, CA (US)

Correspondence Address: MEDTRONIC AVE, INC., 3576 UNOCAL PLACE, SANTA ROSA, CA, 95403, US

(21) Appl. No.: 10/302,646

(22) Filed: Nov. 21, 2002

(51) Int. Cl. A61F 2/06

(52) U.S. Cl. 623/1.11

(57) ABSTRACT

A stent delivery system comprises an inner member and an expandable balloon mounted thereon in a collapsed state. A compressible stent, having a proximal edge and a distal edge, is mounted around the expandable balloon. At least first and second retention bumps are respectively affixed adjacent to the proximal and distal edges of the stent in order to reduce the longitudinal displacement of the compressible stent during insertion and/or retraction of the balloon/stent assembly.

### FIELD OF THE INVENTION

This invention relates generally to an intravascular stent deployment apparatus, and more particularly to a stent delivery apparatus including stent retention members coupled to an expandable balloon adjacent the proximal and distal edges of the stent to retain the stent in position on the balloon during insertion and/or retraction of the balloon/stent assembly.

### BACKGROUND OF THE INVENTION

In a typical percutaneous transluminal coronary angioplasty (PTCA) procedure, a guiding catheter is percutaneously introduced into the cardiovascular system of a patient. The guide catheter is advanced through a vessel until the distal end thereof is at desired location in the vasculature. A guide wire and a dilatation catheter having a balloon on the distal end thereof are introduced into the guiding catheter with the guidewire sliding through the dilatation catheter. The guide wire is first advanced out of the guiding catheter into the patient's coronary vasculature, and the dilatation catheter is then advanced over the previously advanced guide wire until the dilatation balloon is properly positioned across the lesion. Once in position, the flexible, expandable, preformed balloon is inflated to a predetermined size with a liquid or gas at relatively high pressure (e.g. about ten to twelve atmospheres) to radially compress the arthrosclerotic plaque in the lesion against the inside of the artery wall and thereby dilate the lumen of the artery. The balloon is then deflated to a small profile so that the dilatation catheter may be withdrawn from the patient's vasculature and blood flow resumed through the dilated artery.

In angioplasty procedures of the kind described above, there may occur a restenosis of the artery; i.e., a re-narrowing of the treated coronary artery which is related to the development of neo-intinmal hyperplasia that occurs within the artery after it has been treated as described above. In a sense, restenosis is scar tissue that forms in response to mechanical intervention within a vascular structure. To prevent restenosis and strengthen the area, an intravascular prosthesis generally referred to as a stent can be implanted for maintaining vascular patency inside the artery at the lesion. The stent is mounted in a deployment or compressed state around a deflated balloon, and the balloon/stent assembly is maneuvered through a patient's vasculature to the site of a target lesion. The stent is then expanded to a larger diameter for placement or implantation in the vasculature. The stent effectively overcomes the natural tendency of the vessel walls of some patients to close back down, thereby maintaining a normal flow of blood through the vessel that would not be possible if the stent was not in place.

A known expandable stent, which is delivered on a balloon catheter, may be considered to be a stainless steel cylinder having a number of openings in its circumference resulting in a scaffolding when expanded. The stainless steel cylinder is compressed onto the outside of a non-expanded balloon catheter. Unfortunately, the limited amount of securment between the stent and the balloon is not always adequate to ensure that the stent will properly stay in place while advancing the stent to and through a target lesion. Additionally, the outer surface of the delivery device is uneven because the stent generally extends outwardly beyond the balloon. Thus, the stent may contact a vessel wall and be displaced while the catheter negotiates a narrow vessel. Furthermore, during a coronary intervention, the physician may have difficulty crossing the target lesion. In such cases, it may be necessary to pull the stent delivery system back into the guide catheter. Such procedures can cause premature displacement of the stent resulting in serious risk to the patient.

For example, the guide catheter is generally inserted through the abdominal aorta to a point just beyond the ostium, the location from which the right coronary artery and the left main artery diverge. Blockages or lesions may form in smaller coronary vessels, and medical practitioners may sometimes predilatate the target area as, for example, by balloon angioplasty. Sometimes, however, predilatation is not performed, and doctors proceed directly to a primary stenting procedure. In such cases, there are occasions when the balloon/stent catheter cannot be properly positioned within the target area due to the constriction of the vessel and must be retracted back into the guide catheter. Even when predilatation is performed, vascular spasms and/or reclosure of the vessel may occur rendering it difficult to properly align the balloon/stent assembly likewise requiring retraction into the guide catheter. In addition, the lesion may be heavily calcified requiring a high insertion pressure. In either case, unwanted displacement of the compressed stent may occur.

It should therefore be appreciated that it would be desirable to provide a low profile stent delivery and deployment apparatus that provides for increased retention force of the compressed stent on the balloon.

### SUMMARY OF THE INVENTION

According to an aspect of the invention, there is a provided a stent delivery system comprising an inner member and an expandable balloon mounted thereon in a collapsed state. A compressible stent, having a proximal edge and a distal edge, is mounted around the expandable balloon. At least first and second retention bumps are respectively affixed adjacent to the proximal and distal edges of the stent in order to reduce the longitudinal displacement of the compressible stent during insertion and/or retraction of the balloon/stent assembly.

### BRIEF DESCRIPTION OF THE DRAWINGS

The following drawings are illustrative of particular embodiments of the invention and therefore do not limit the scope of the invention, but are presented to assist in providing a proper understanding. The drawings are not to scale (unless so stated) and are intended for use in conjunction with the explanations in the following detailed descriptions. The present invention will hereinafter be described in conjunction with the appended drawings, wherein like reference numerals denote like elements, and;

FIG. 1 is a longitudinal view of a stent and balloon assembly in accordance with the present invention that utilizes generally hemispherical stent retention members coupled to the balloon adjacent the proximal and distal edges of the stent; and

FIG. 2 is a cross-sectional view of the balloon/stent assembly shown in FIG. 1 taken along line 2-2.

### DETAILED DESCRIPTION OF THE EXEMPLARY EMBODIMENT

The following description is exemplary in nature and is not intended to limit the scope, applicability, or configuration of the invention in any way. Rather, the following description provides a convenient illustration for implementing an exemplary embodiment of the invention. Various changes to the described embodiment may be made in the function and arrangement of the elements described herein without departing from the scope of the invention.

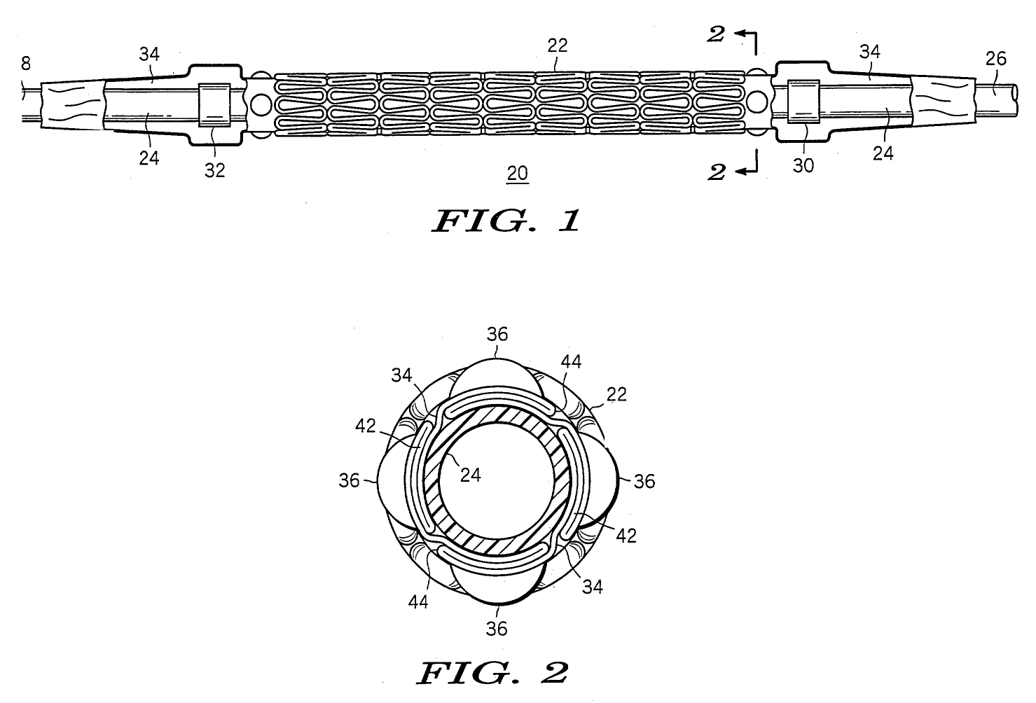

FIG. 1 is a longitudinal, cross-sectional view of a balloon/stent assembly embodying the principles of the present invention. The balloon/stent assembly shown generally at 20 comprises a stent 22, an inner member or wire lumen 24 having a distal end 26 and a proximal end 28, and distal and proximal radiopaque marker bands 30 and 32 respectively positioned on inner member or wire lumen 24 near the distal and proximal ends of stent 22 respectively. Stent 22 may be of any form or configuration suitable for the intended purpose, and may comprise one or more stent segments depending on the size and configuration of the vessel to be treated. It will be recognized by those skilled in the art that inner member or wire lumen 24 is configured for the insertion of a conventional guide wire (not shown) which will enable the balloon/stent assembly to be guided and positioned at a target location in the vessel to be treated.

Any conventional or modified balloon catheter device may be used such as a PTCA balloon catheter. An expandable balloon portion 34 is mounted on inner member 24 in a compressed or collapsed state beneath stent 22 and extends beyond the proximal and distal ends of stent 22. Balloon 34 is generally made of a pliable material such as polyethylene, polyethylene terathalate, PEBAX (polyamide block copolymers and polyester block copolymers), polyvinyl chloride, polyolefin, nylon or the like. The length and the diameter of the balloon may be selected to accommodate the particular configuration of the stent to be deployed. Stent 22 may be constructed of any implantable material having good mechanical strength, such as stainless steel, titanium, tantalum, super-elastic nickel-titanium alloys, or high-strength thermoplastic polymers. The outside of the stent may be selectively plated with platinum or other implantable radiopaque substance to provide visibility during fluoroscopy. The cross-sectional shape of the finished stent 22 maybe circular, ellipsoidal, rectangular, hexagonal, square, or any other desired shape, although a circular or ellipsoidal cross-section is preferable. The length and width of stent 22 is generally determined to a large degree by the size of the vessel into which the stent will be deployed. It must be of sufficient length to maintain its axial orientation without shifting under the hydraulics of blood flow and extend across a significant portion of the target area, while at the same time not be unnecessarily long so as to result in the introduction of an unnecessarily large amount of material into the vessel.

After stent selection, the stent 22 is compressed upon the outside of balloon 34. An inner sheath (not shown) is placed over each end of balloon 34 and an exterior sheath (also not shown), is placed over the ends of the interior sheath so as to cover stent 22 and overlap with the interior sheaths. The assembly is then pressurized by introducing air or an inert gas such as nitrogen through the lumen 24 into the interior of balloon 34 so as to expand the balloon within the sheaths. The assembly is then heated while maintaining pressurization of the balloon. The pressure may be, for example, approximately 70 psi and the temperature approximately 150 degrees Fahrenheit. Following heating, the balloon/stent assembly is allowed to cool within the sheaths thereby setting the shape of balloon 34. The sheaths may then be removed. This process is described in detail in U.S. Pat. No. 5,836,965 entitled “Stent Delivery and Deployment Method” issued Nov. 17, 1998, the teachings of which are hereby incorporated by reference.

Marker bands 30 and 32, which may be viewed through fluoroscopy, assist in positioning the assembly. When the assembly is properly located across a lesion, the balloon may be inflated in a conventional manner. This results in the general uniform, symmetrical expansion of the stent and balloon. The amount of inflation and thus the amount of expansion of the stent may be varied as dictated by the lesion itself.

To prevent displacement of stent 22 on balloon 34 during insertion or retraction of the balloon/stent assembly shown in FIG. 1, bumps 36 are provided on balloon 34, preferably prior to crimping stent 22 around balloon 34. For example, a plurality of such bumps is shown disposed around the periphery of balloon 34 substantially adjacent to the proximal and distal edges of stent 22. Bumps 36 adhere to balloon 34 and may be made of a liquid/moldable elastomeric adhesive material such as silicone adhesive. Bumps 36 may be positioned, for example, between 0.5 mm and 0.75 mm from the distal and proximal ends of stent 22. The height of bumps 36 should be sufficient such that when fixed to balloon 34, they prevent or block stent 22 from moving longitudinally on balloon 34 more than the distance between bumps 36 and the respective adjacent stent edge. Preferably, bumps 36 have a height substantially equal to the thickness of the sent wall. In a preferred embodiment, bumps 36 are generally hemispherical in shape and are equally spaced around the periphery of balloon 34; however, other shapes and spacings may be utilized as necessary.

As stated previously, retention bumps 36 may be made of an elastomeric material such as silicone. If desired, a radiopaque material may be added to the elastomeric material to render the bumps visible by means of fluoroscopy. For example, a radiopaque material such as barium sulfate or tantalum may be added to the silicone. The use of radiopaque retention bumps could, if desired, eliminate the need for marker bands 30 and 32.

FIG. 2 is a cross-sectional view of the balloon/stent assembly shown in FIG. 1 taken along line 2-2. As can be seen, balloon 34 is collapsed upon inner member 24 such that a plurality of folds (in this case four) 42 is produced. Each fold 42 has a longitudinal edge 44. The wings or folds 42 of balloon 34 are formed by pulling the balloon catheter through a forming tool having a generally cylindrical cross-section and defining a terminal opening configured to produce the desired number of wings or folds in the balloon. For example, the terminal opening may include four slits extending radially outward from the end of the forming tool, the number of slits depending upon the number of folds to be produced. As the balloon catheter is pulled through the forming tool, the balloon is pushed through the terminal opening and exits having, for example, four separate flutes. The balloon catheter bearing the fluted balloon portion is then pulled into a sheath, preferably a two-part sheath made of Teflon or other suitable material so that the flutes fold and wrap around the catheter in a clockwise direction to form a generally spiral configuration. The sheath/balloon catheter assembly is then heated, preferably by placing the assembly in an oven, to form a crease in substantially the length of each of the folded flutes. Following heating, balloon 34 retains the creases formed in the wings to define a generally symmetrical, cylindrical cross-section as can be seen in FIG. 2.

Referring still to FIG. 2, it can be seen that four folds 42 have been formed in balloon 34 each having an edge 44. It should be appreciated, however, that the number of folds 42 might be varied to accommodate different configurations and/or applications. Retaining bumps 36 are bonded or otherwise adhesively coupled to balloon 34, preferably proximate edges 44 as shown. A variety of adhesives is suitable for this purpose; e.g. ultra-violet cure, instant cure, epoxy type, cyanoacrylate type, etc. After bumps 36 are positioned and secured on balloon 34, stent 22 may be crimped or compressed onto balloon 34. Again, it should be appreciated that while four retaining bumps 36 have been shown, the number of retaining bumps may be varied to achieve a desired configuration and retention capability.

Thus, there has been provided an intravascular support device including an expandable balloon having a stent compressed thereon and one or more retention bumps affixed to the balloon adjacent to the proximal and distal ends of the stent. This enhances stent retention on the balloon thus minimizing unwanted longitudinal displacement of the stent during insertion and/or retraction of the balloon/stent assembly.

In the foregoing specification, the invention has been described with reference to a specific embodiment. It should be appreciated, however, that various modifications and changes might be made without departing from the scope of the invention as set forth in the appended claims. Accordingly, the specification and figures should be regarded as illustrative rather than restrictive, and all such modifications are intended to be included within the scope of the present invention.

1. A stent delivery system, comprising:
an inner member;
an expandable balloon mounted in a collapsed state on said inner member;
a compressible stent mounted in a compressed state around said expandable balloon, said compressible stent having a proximal edge and a distal edge; and
at least first and second retention bumps affixed to said balloon substantially adjacent said proximal edge and said distal edge respectively for reducing longitudinal displacement of said compressible stent.

2. A stent delivery system according to claim 1 wherein said at least first and second retention bumps comprise first and second pluralities of retention bumps affixed around the periphery of said expandable balloon substantially adjacent said proximal edge and said distal edge respectively.

3. A stent delivery system according to claim 2 wherein each retention bump in each of said first and second pluralities of retention bumps is substantially hemispherical in shape.

4. A stent delivery system according to claim 2 wherein each of said first and second pluralities of retention bumps are substantially equally spaced around the periphery of said balloon.

5. A stent delivery system according to claim 4 wherein said expandable balloon comprises a plurality of folds around said inner member, each fold having an edge, and wherein each of said retention bumps in said first and second pluralities of retention bumps is positioned proximate the edge of one of said plurality of folds.

6. A stent delivery system according to claim 3 wherein each retention bump of said first and second pluralities of retention bumps has a height at least equal to the thickness of said stent.

7. A stent delivery system according to claim 6 wherein each of said retention bumps is adhesively coupled to said expandable balloon.

8. A stent delivery system according to claim 7 wherein each of said pluralities of retention bumps is made of an elastomeric material.

9. A stent delivery system according to claim 8 wherein said elastomeric material is silicone.

10. A stent delivery system according to claim 6 wherein each of said first and second pluralities of retention bumps is positioned approximately 0.5 mm-0.75 mm from said proximal edge and said distal edge respectively.

11. A stent delivery system according to claim 2 wherein said retention bumps are radiopaque.

12. A stent delivery system according to claim 8 wherein said elastomeric material contains a radiopaque substance.

13. A stent delivery system according to claim 12 wherein said radiopaque substance is barium sulfate.

14. A stent delivery system according to claim 12 wherein said radiopaque substance is tantalum.
